(19) United States
(12) Patent Application Publication
(10) Pub. No.: US 2023/0270488 A1
(43) Pub. Date: Aug. 31, 2023

(54) MEDICAL DEVICE FOR TREATING ESOPHAGEAL ATRESIA

(71) Applicant: BOSTON SCIENTIFIC SCIMED INC., MAPLE GROVE, MN (US)

(72) Inventors: RONALD L. LANCASTER, HOPKINTON, MA (US); MANDI ROUTON, MINNEAPOLIS, MN (US); JACOB HEINZ, MAPLE GROVE, MN (US); AIDEN FLANAGAN, Kilcolgan (IE)

(73) Assignee: BOSTON SCIENTIFIC SCIMED INC., MAPLE GROVE, MN (US)

(21) Appl. No.: 18/114,534

(22) Filed: Feb. 27, 2023

Related U.S. Application Data
(60) Provisional application No. 63/314,946, filed on Feb. 28, 2022.

(51) Int. Cl. A61B 18/14

(52) U.S. Cl. CPC A61B 18/1492; A61B 2018/0022

(57) ABSTRACT

A medical device for treating esophageal atresia may include a handle, an elongate shaft extending distally from the handle, the elongate shaft configured to extend into an esophagus of a patient and into an upper esophageal pouch of the patient, the elongate shaft having a distal nub configured to engage a distal end of the upper esophageal pouch, a force absorbing member disposed within the handle, the force absorbing member permitting the elongate shaft to translate axially relative to the handle, and an inflatable balloon disposed within the handle. An internal pressure within the inflatable balloon increases as the force absorbing member is compressed.

### CROSS-REFERENCE TO RELATED APPLICATIONS

This application claims the benefit of U.S. Provisional Patent Application Ser. No. 63/314,946, filed Feb. 28, 2022, which is incorporated herein by reference.

### TECHNICAL FIELD

The present disclosure pertains to medical devices, and methods for using medical devices. More particularly, the present disclosure pertains to medical devices for treating esophageal atresia.

### BACKGROUND

Esophageal atresia is a condition where individuals are born with an incomplete esophagus which does not connect the throat to the stomach. The incomplete esophagus may form pouches having a closed end. Current treatment procedures, for example the Foker Technique, are very invasive and may cause extreme trauma. In some cases, individuals are chemically paralyzed to prevent motion for several weeks which may cause weight loss and muscle wasting, reduced brain size, and/or reduced bone density. Additionally, the Foker Technique requires a minimum body weight of 3.5 kilograms, which further limits its use in some situations such as a premature birth. The esophagus naturally grows in response to stretch as the individual grows, so the tissue “wants” to grow. The Foker Technique uses sutures which are surgically attached to both pouch ends of the esophagus and the stomach, passed through the skin on the patients back. The sutures are periodically pulled a small amount, inducing stretch on the esophagus causing growth over time. During that time, the individuals remain paralyzed to prevent suture and/or tissue rupture. Another shortcoming of this procedure is that the applied forces are uncontrolled and suture attachments to the pouches cause high stress concentrations leading to significant tissue morbidity that requires removal prior to anastomosis (surgical connection of the two ends to form a complete esophagus). In another alternative, magnets are implanted into each of the pouch ends of the esophagus and their attraction to each other applies force to the tissue and stretches the pouch ends of the esophagus towards each other over time. However, the force is non-linear and uncontrolled. Therefore, a minimum distance of 4 cm or less is required to have sufficient attraction and as the distance between the magnets decreases, the force applied to the tissue by the magnets increases, which may lead to rupture and/or additional tissue damage. Additionally, long-term sedation and/or immobilization, removal of mucous and/or saliva via suction, and alternative feeding procedures are still required, as is a surgical procedure to later remove the magnets and connect the pouch ends. As such, there is an ongoing need to provide alternative medical devices and procedures for treating esophageal atresia.

### SUMMARY

In one example, a medical device for treating esophageal atresia may comprise a handle; an elongate shaft extending distally from the handle, the elongate shaft configured to extend into an esophagus of a patient and into an upper esophageal pouch of the patient, the elongate shaft having a distal nub configured to engage a distal end of the upper esophageal pouch; a force absorbing member disposed within the handle, the force absorbing member permitting the elongate shaft to translate axially relative to the handle; and an inflatable balloon disposed within the handle. An internal pressure within the inflatable balloon increases as the force absorbing member is compressed.

In addition or alternatively to any example described herein, the inflatable balloon cooperates with the force absorbing member to exert a force along the elongate shaft to apply the force to the distal end of the upper esophageal pouch.

In addition or alternatively to any example described herein, the medical device may further comprise a balloon shaft fixedly attached to the inflatable balloon and having an inflation lumen extending from the inflatable balloon to a regulator disposed outside of the handle.

In addition or alternatively to any example described herein, the regulator is configured to deflate the inflatable balloon when the internal pressure within the inflatable balloon exceeds a preset pressure limit.

In addition or alternatively to any example described herein, the handle includes an adjustment mechanism configured to change the internal pressure within the inflatable balloon.

In addition or alternatively to any example described herein, the adjustment mechanism is configured to change the force applied to the distal end of the upper esophageal pouch.

In addition or alternatively to any example described herein, the adjustment mechanism includes a knob configured to rotate relative to the handle, and a threaded shaft engaged with the knob within the handle. A first end of the threaded shaft engages a first end of a tubular retainer, the force absorbing member and the inflatable balloon being disposed within the tubular retainer. Rotation of the knob relative to the handle translates the tubular retainer axially within the handle.

In addition or alternatively to any example described herein, the adjustment mechanism is configured to axially translate the inflatable balloon within the handle.

In addition or alternatively to any example described herein, the adjustment mechanism is configured to axially translate the force absorbing member within the handle.

In addition or alternatively to any example described herein, the medical device may further comprise an elongate tubular member extending distally from the handle. The distal nub has a first outer diameter. The elongate tubular member has a second outer diameter less than the first outer diameter. At least a portion of the elongate shaft is movably disposed within the elongate tubular member.

In addition or alternatively to any example described herein, the elongate tubular member includes a suction lumen configured to be in fluid communication with a source of suction.

In addition or alternatively to any example described herein, the elongate tubular member includes a distal basket configured to receive at least a portion of the distal nub. The distal basket is configured such that suction within the distal end of the upper esophageal pouch is permitted regardless of a position of the distal nub relative to the distal basket.

In addition or alternatively to any example described herein, a medical device for treating esophageal atresia may comprise a handle; an elongate shaft extending distally from the handle, the elongate shaft configured to extend into an esophagus of a patient and into an upper esophageal pouch of the patient, the elongate shaft having a distal nub configured to engage a distal end of the upper esophageal pouch; a spring disposed within the handle, the spring permitting the elongate shaft to translate axially relative to the handle in response to movements of the patient; and an inflatable balloon disposed within the handle. An internal pressure within the inflatable balloon increases as the spring is compressed.

In addition or alternatively to any example described herein, a balloon shaft fixedly attached to the inflatable balloon and including an inflation lumen extending from the handle engages with a longitudinal advancement-retraction mechanism disposed proximal of the handle.

In addition or alternatively to any example described herein, the handle includes a knob configured to rotate relative to the handle to change the internal pressure within the inflatable balloon.

In addition or alternatively to any example described herein, the handle includes a position indicator configured to communicate an axial position of the elongate shaft relative to the handle.

In addition or alternatively to any example described herein, a method of treating esophageal atresia may comprise preparing a medical device for treating esophageal atresia. The medical device includes a handle including an adjustment mechanism, an elongate shaft extending distally from the handle, a force absorbing member disposed within the handle and permitting the elongate shaft to translate axially relative to the handle, and an inflatable balloon disposed within the handle. The elongate shaft has a distal nub configured to engage a distal end of an upper esophageal pouch of the patient. The method further includes inflating the inflatable balloon to an internal pressure, wherein the internal pressure is an initial pressure, positioning the distal nub of the elongate shaft against the distal end of the upper esophageal pouch, fixing the medical device in place relative to the distal end of the upper esophageal pouch, and actuating the adjustment mechanism to change the internal pressure within the inflatable balloon to a treatment pressure. The inflatable balloon cooperates with the force absorbing member to exert a force along the elongate shaft to apply the force to the distal end of the upper esophageal pouch. The force absorbing member is configured to permit the elongate shaft to translate axially relative to the handle in response to patient movements while maintaining the distal nub against the distal end of the upper esophageal pouch.

In addition or alternatively to any example described herein, the internal pressure within the inflatable balloon increases as the force absorbing member is compressed in response to patient movements.

In addition or alternatively to any example described herein, an inflation lumen extends from the inflatable balloon to a regulator disposed outside of the handle. The regulator is configured to deflate the inflatable balloon when the internal pressure within the inflatable balloon exceeds a preset pressure limit.

In addition or alternatively to any example described herein, the method may further comprise monitoring the internal pressure within the inflatable balloon over time, monitoring axial translation of the elongate shaft over time, and actuating the adjustment mechanism to increase the internal pressure when the internal pressure drops below a predetermined pressure threshold or when axial translation of the elongate shaft reaches a predetermined translation threshold.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates aspects of esophageal atresia;

FIGS. 2-3 illustrate selected aspects of a medical device for treating esophageal atresia;

FIGS. 4-5 illustrate selected features disposed proximate a distal end of the medical device of FIGS. 2-3;

FIG. 6 is partial cross-sectional views illustrate selected aspects disposed within a handle of the medical device of FIGS. 2-3;

FIG. 7 is partial a cross-sectional view illustrating selected aspects of an inflatable balloon disposed within the handle of the medical device of FIGS. 2-3 in a deflated configuration;

FIG. 8 is partial a cross-sectional view illustrating selected aspects of the inflatable balloon disposed within the handle of the medical device of FIGS. 2-3 in an inflated configuration;

FIG. 9 illustrates selected aspects of the medical device of FIGS. 2-3 as viewed from the opposite side compared to FIG. 2;

FIG. 10 illustrates selected aspects of a pressure control of the medical device of FIGS. 2-3;

FIG. 11 is a block diagram illustrating selected aspects of a pre-placement procedure associated with the medical device of FIGS. 2-3;

FIG. 12 is a block diagram illustrating selected aspects of a placement procedure associated with the medical device of FIGS. 2-3; and

FIGS. 13-19 illustrate selected aspects related to using the medical device of FIGS. 2-3.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Some features and/or reference numbers may not be shown in each figure for clarity. Similarly, each and every element and/or reference number may not be discussed in detail with respect to each figure. Some reference numbers and/or features may be shown and/or described in other figures in more detail, and those reference numbers and/or features may be shown and/or identified in some figures merely for reference.

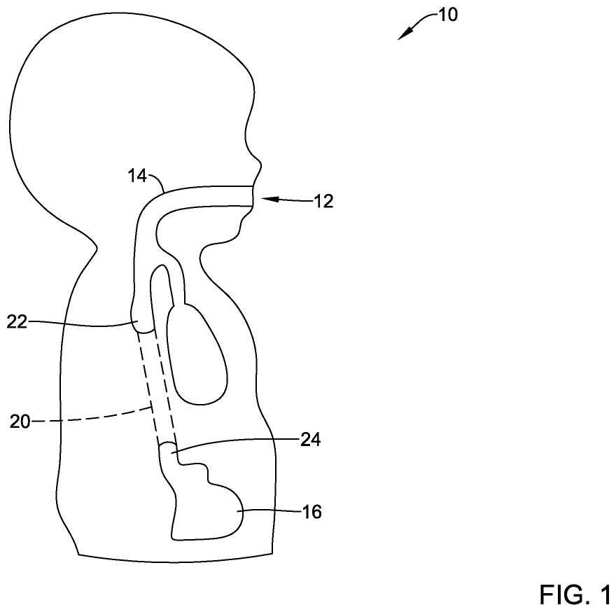

FIG. 1 illustrates aspects of an example of esophageal atresia, which is a congenital defect affecting a number of babies each year. As shown in FIG. 1, a patient 10 may be born with an esophagus 20 that is incomplete, resulting in the patient 10 having an upper portion 14 of the esophagus 20 extending from the mouth 12 to an upper esophageal pouch 22 and a lower esophageal pouch 24 connected to the stomach 16. The upper esophageal pouch 22 and the lower esophageal pouch 24 may be spaced apart from each other within the torso of the patient 10, such that the upper esophageal pouch 22 is unconnected to or discontinuous with the lower esophageal pouch 24. This disclosure describes medical devices and/or systems for treating pediatric esophageal atresia without subjecting the patient 10 to or with reduced long-term sedation and/or immobilization.

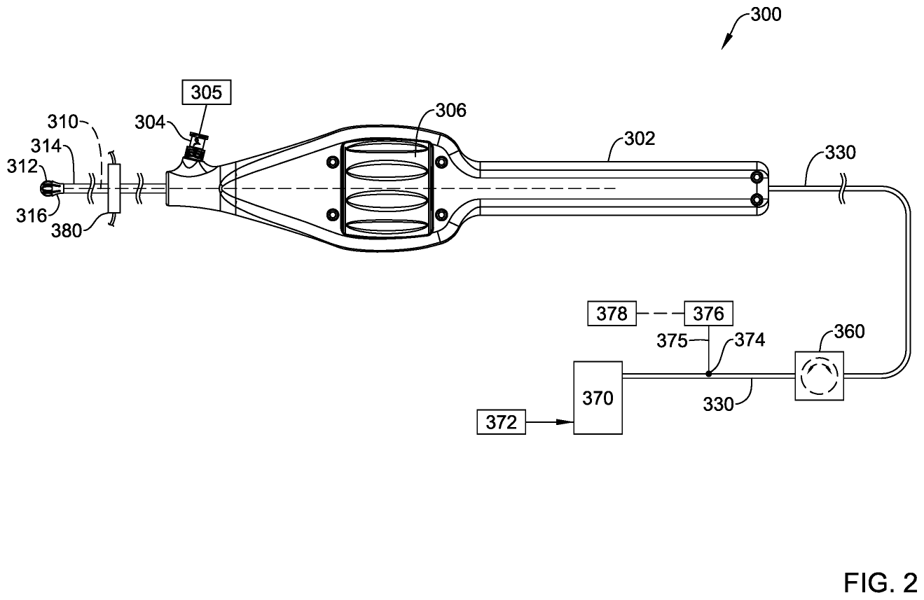

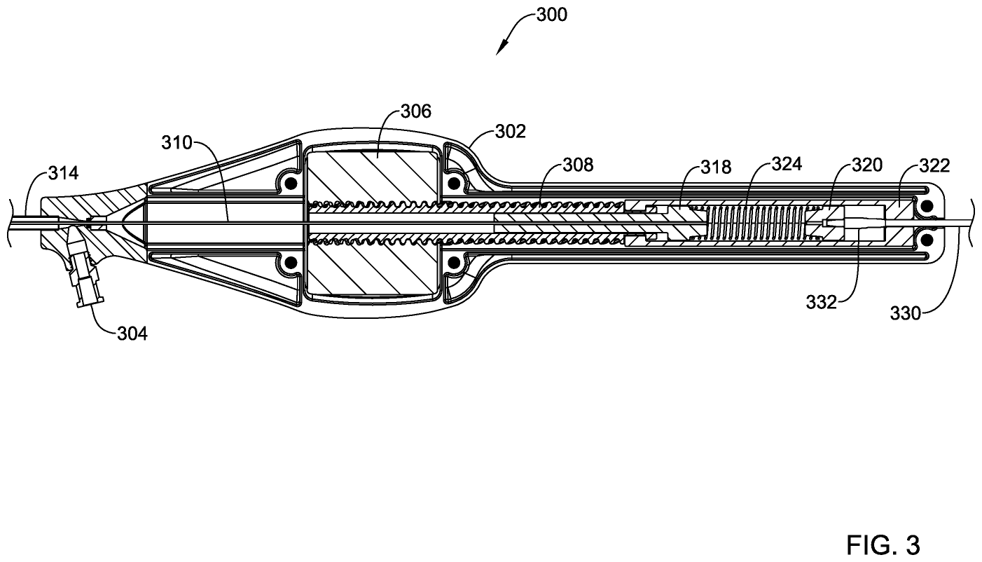

FIG. 2 is a side view and FIG. 3 is a partial cross-sectional view illustrating selected aspects of a medical device 300 for treating esophageal atresia. The medical device 300 may include a handle 302. In some embodiments, the handle 302 may be a generally elongate structure having and/or capable of having elements disposed therein. Other configurations are also contemplated. Some suitable, but non-limiting, examples of materials for the handle 302, including but not limited to polymeric materials, metallic materials, and/or composite materials, are discussed below.

The medical device 300 may include an elongate shaft 310 extending distally from the handle 302. In some embodiments, the elongate shaft 310 may be at least partially disposed within the handle 302. The elongate shaft 310 may be configured to extend through the mouth 12 of the patient 10, into the upper portion 14 of the esophagus 20, to the upper esophageal pouch 22 of the patient 10. The elongate shaft 310 may include a distal nub 312 configured to engage a distal end of the upper esophageal pouch 22. In at least some embodiments, the distal nub 312 of the elongate shaft 310 may have a first outer diameter. In some embodiments, the first outer diameter of the distal nub 112 may be about 14 French (Fr) (about 4.67 millimeters (mm)), about 16 Fr (about 5.33 mm), about 18 Fr (about 6.0 mm), about 20 Fr (about 6.67 mm), about 22 Fr (about 7.33 mm), or another suitable size depending on the size of the patient 10. The distal nub 312 may have a fixed size in which the first outer diameter is the maximum outer diameter of the distal nub 312. In some alternative embodiments, the distal nub 312 may have a variable size and/or the distal nub 312 may be configured to change size in situ. In such an instance, the first outer diameter may be the maximum outer diameter of the distal nub 312 when expanded.

In some embodiments, the medical device 300 may include a plurality of distal nubs detached from the elongate shaft 310. In some embodiments, the plurality of distal nubs may include at least two distal nubs, at least three distal nubs, at least four distal nubs, etc. The plurality of distal nubs may each have a different size (e.g., maximum outer diameter). For instance, the medical device 300 may be provided with a set of distal nubs of a range of sizes (e.g., about 16 Fr, about 20 Fr, about 22 Fr, etc.). In some embodiments, the medical device 300 may be packaged as a part of a kit including the plurality of distal nubs. During preparation for use (e.g., prior to insertion into the patient), the user may select the desired size for the distal nub 312, based on the patient's age, weight, size, esophageal dimensions, shape of the upper esophageal pouch 22, etc., from the plurality of distal nubs and then attach the distal nub 312 to a distal end of the elongate shaft 310, as described herein.

In some embodiments, the distal nub 312 may be configured to engage the distal end of the upper esophageal pouch 22 in a manner that substantially prevents cutting, tearing, abrasion, etc. of the tissue of the upper esophageal pouch 22. In some embodiments, the distal nub 312 may have a generally bulbous shape. In some embodiments, the distal nub 312 may have a generally spherical shape. In some embodiments, the distal nub 312 may have a generally rounded shape (e.g., ovular, egg-shaped, a non-spherical rounded shape, etc.). In some embodiments, the distal nub 312 may be solid. In some embodiments, the distal nub 312 may be hollow. In some embodiments, the distal nub 312 may be formed from a generally soft and/or compliant material. In some embodiments, the distal nub 312 may be formed from silicone or another polymer. Other materials and/or configurations are also contemplated. Some suitable, but non-limiting, examples of materials for the distal nub 312, including but not limited to polymeric materials, metallic materials, and/or composite materials, are discussed below.

In some embodiments, the distal nub 312 may include a pressure sensor and/or a force sensor disposed therein. In some embodiments, the pressure sensor and/or the force sensor may be configured to measure a force and/or pressure applied to the distal end of the upper esophageal pouch 22 by the distal nub 312. The pressure sensor and/or the force sensor may include a wire, a fiber optic wire, etc. extending within and/or through the elongate shaft 310 and/or the handle 302. In some embodiments, the pressure sensor and/or the force sensor may be operably connected to a monitoring device, such as and/or which may include a display, a computer, etc. Other configurations are also contemplated.

In some embodiments, the elongate shaft 310 may extend axially into and/or through the handle 302. The elongate shaft 310 may be generally flexible in a lateral direction to permit navigation through the mouth 12 of the patient 10 into the upper portion 14 of the esophagus 20 and/or the upper esophageal pouch 22. The elongate shaft 310 may be generally rigid and/or stiff in an axial direction to promote pushability and/or transmission of force axially along the elongate shaft 310, as discussed herein. In some embodiments, the elongate shaft 310 may have a tubular elongate structure having at least one lumen extending therethrough. In some embodiments, the elongate shaft 310 may have a solid elongate structure. Other configurations, including combinations thereof, are also contemplated. Some suitable, but non-limiting, examples of materials for the elongate shaft 310, including but not limited to polymeric materials, metallic materials, and/or composite materials, are discussed below.

In some embodiments, the medical device 300 may include an elongate tubular member 314 extending distally from the handle 100. In at least some embodiments, the elongate tubular member 314 may be configured to extend through the mouth 12 of the patient 10 and into the upper esophageal pouch 22 of the patient 10. At least a portion of the elongate shaft 310 may be movably disposed within at least a portion of the elongate tubular member 314. The elongate tubular member 314 may have a second outer diameter less than the first outer diameter of the distal nub 312. In some embodiments, the second outer diameter may be about 6 French (Fr) (about 2.0 millimeters (mm)), about 8 Fr (about 2.67 mm), about 10 Fr (about 3.33 mm), or about 12 Fr (4.0 mm).

In some embodiments, the medical device 300 may permit separate support of respiration by other devices alongside and/or while the medical device 300 is in place without impeding respiratory treatment. For example, in some embodiments, the elongate tubular member 314 may be sized to permit intubation alongside the elongate tubular member 314. In some alternative embodiments, the second outer diameter of the elongate tubular member 314 may be of sufficient size to accommodate a respiration tube disposed within the elongate tubular member 314 along with the elongate shaft 310.

In some embodiments, the medical device 300 and/or the elongate tubular member 314 may be configured to irrigate the distal end of the upper esophageal pouch 22. In some embodiments, the medical device 300 and/or the elongate tubular member 314 may be configured for suction of mucous and/or saliva from the distal end of the upper esophageal pouch 22. In some embodiments, the medical device 300 and/or the elongate tubular member 314 may be simultaneously used for irrigation of the distal end of the upper esophageal pouch 22 and suction of mucous and/or saliva from the distal end of the upper esophageal pouch 22.

In some embodiments, the elongate tubular member 314 may include an irrigation lumen and/or the second outer diameter of the elongate tubular member 314 may be of sufficient size to accommodate an irrigation tube disposed within the elongate tubular member 314 along with the elongate shaft 310. In some embodiments, the elongate tubular member 314 may include a suction lumen and/or the second outer diameter of the elongate tubular member 314 may be of sufficient size to accommodate a suction tube disposed within the elongate tubular member 314 along with the elongate shaft 310. In some embodiments, the elongate tubular member 314 may include an irrigation lumen and a suction lumen, and/or the second outer diameter of the elongate tubular member 314 may be of sufficient size to accommodate an irrigation tube and a suction tube disposed within the elongate tubular member 314 along with the elongate shaft 310. Some suitable, but non-limiting, examples of materials for the elongate tubular member 314, including but not limited to polymeric and/or metallic materials, are discussed below.

The medical device 300 and/or the handle 302 may include a side port 304. The side port 304 may be in fluid communication with the elongate tubular member 314 and/or the suction lumen. In some embodiments, the side port 304 may be in fluid communication with a fluid management system 305. In some embodiments, the fluid management system 305 may include an irrigation system (e.g., including a source of irrigation fluid, etc.), a suction system (e.g., including a source of suction or a vacuum source, etc.), or any combination thereof. In some embodiments, the medical device 300 and/or the handle 302 may include a plurality of ports in fluid communication with the elongate tubular member 314 and/or the fluid management system 305. In some embodiments, the plurality of ports may each be in fluid communication with a different portion of the fluid management system 305. Other configurations are also contemplated.

In some embodiments, the side port 304 may be used in cooperation with the lumen of the elongate tubular member 314 to irrigate the distal end of the upper esophageal pouch 22. In at least some embodiments, the side port 304 may be used in cooperation with the elongate tubular member 314 for suction of fluid, mucous, debris, etc. from the distal end of the upper esophageal pouch 22. Other configurations are also contemplated.

In some embodiments, the medical device 300 may be configured to treat esophageal atresia and for suction of mucous and/or saliva from the distal end of the upper esophageal pouch 22 simultaneously, thereby preventing mucous and/or saliva from entering the trachea, which could lead to potential disruption of adequate respiration. As such, the medical device 300 is able to help sustain the life of the patient while treating the esophageal atresia.

In some embodiments, irrigation of the distal end of the upper esophageal pouch 22 may be periodic and/or performed as needed. In some embodiments, suction of fluid, mucous, saliva, and/or debris from the distal end of the upper esophageal pouch 22 may be continuous. Other configurations are also contemplated.

In some embodiments, the medical device 300 and/or the elongate tubular member 314 may be fixed in place relative to the patient 10 using a fixation element 380. In some embodiments, the fixation element 380 may be secured to the face of the patient 10, such as with adhesive tape. In some embodiments, the fixation element 380 may include a headgear or a mouthpiece secured to the head of the patient 10. In some embodiments, the fixation element 380 may include a mask or other structure configured to at least partially surround and/or encompass the head of the patient 10. In some embodiments, the medical device 300 and/or the elongate tubular member 314 may be secured to the patient 10 and/or may be secured relative to the patient 10, the mouth 12, and/or the distal end of the upper esophageal pouch 22 by securing the medical device 300 and/or the elongate tubular member 314 to another medical device or product such as a NeoBar® from Neotech Products. Other suitable medical devices and/or products are also contemplated.

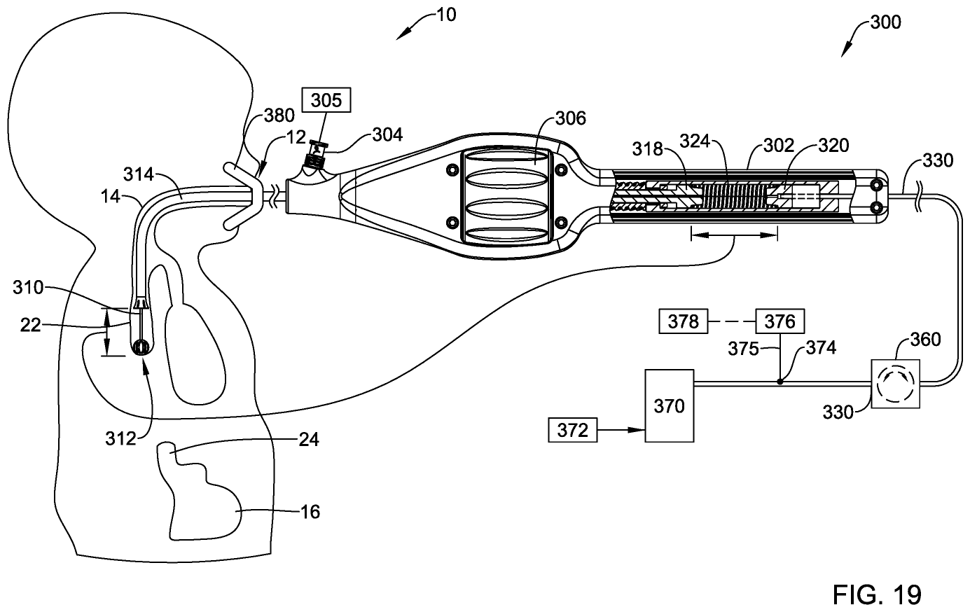

In some embodiments, the medical device 300 may include a force absorbing member 324 disposed within the handle 302, as seen in FIG. 3 for example. The force absorbing member 324 may be configured to permit the elongate shaft 310 to translate axially relative to the handle 302. In some embodiments, the force absorbing member 324 may be configured to permit the elongate shaft 310 to translate axially relative to the handle 302 in response to movements of the patient 10 (e.g., FIG. 19). In some embodiments, the force absorbing member 324 may be configured to exert a force along the elongate shaft 310 to apply the force to the distal end of the upper esophageal pouch 22 of the patient 10. In some embodiments, the force absorbing member 324 may be configured to permit the elongate shaft 310 to translate axially relative to the handle 302 while maintaining the distal nub 312 and/or the force against the distal end of the upper esophageal pouch 22. In some embodiments, the elongate shaft 310 may be fixedly secured relative to a distal end of the force absorbing member 324.

In some embodiments, the force may be applied constantly to the distal end of the upper esophageal pouch 22 of the patient 10. In some embodiments, the force may be varied over time. In some embodiments, the force may be applied cyclically to the distal end of the upper esophageal pouch 22 of the patient 10. For example, in some embodiments, the force may be applied to the distal end of the upper esophageal pouch 22 of the patient 10 for a first interval of time and the force may be removed for a second interval of time, wherein the first interval of time alternates with the second interval of time. In some embodiments, the force may be applied to the distal end of the upper esophageal pouch 22 of the patient 10 at an original value until a certain amount of growth of the upper esophageal pouch 22 occurs and/or the force drops below a certain amount from the original value, and then the force may be increased and/or re-applied back to the original value. In some embodiments, the force may be applied to the distal end of the upper esophageal pouch 22 of the patient 10 such that the force follows a cyclical wave (e.g., a sine wave, etc.), wherein the force is slowly increased over time and then slow decreased over time in a repeating fashion and/or pattern. Other configurations are also contemplated. In some embodiments, the cyclical application of the force may allow and/or permit at least some tissue relaxation and/or reperfusion during the duration of the process and/or procedure. In some embodiments, the force may be applied randomly to the distal end of the upper esophageal pouch 22 of the patient 10 as the patient 10 moves. In some embodiments, the force may vary randomly about a target force value (e.g., the original force). In some embodiments, the force may be applied randomly to the distal end of the upper esophageal pouch 22 of the patient 10 about the target force value with a preset and/or user set maximum force value. Other configurations, including various combinations thereof, are also contemplated. In some embodiments, the force may be no more than 1.0 pounds of force. In some embodiments, the force may be no more than 0.8 pounds of force. In some embodiments, the force may be no more than 0.6 pounds of force. In some embodiments, the force may be no more than 0.5 pounds of force. Other configurations are also contemplated.

In some embodiments, the force absorbing member 324 may include and/or may be a spring. In some embodiments, the force absorbing member 324 may include and/or may be a compression spring. In some embodiments, the force absorbing member 324 may include and/or may be a torsion spring. In some embodiments, the force absorbing member 324 may include and/or may be a spring held in tension. In some embodiments, the force absorbing member 324 may include and/or may be a fluid or a gas. In some embodiments, the force absorbing member 324 may include and/or may be an elastic member. In some embodiments, the force absorbing member 324 and/or the spring may be compressible and/or may be resiliently compressible. In some embodiments, the force absorbing member 324 and/or the spring may be axially compressible and/or may be axially resilient. Other configurations are also contemplated.

In some embodiments, the force absorbing member 324 and/or the spring may be movably disposed within the handle 302. In some embodiments, the force absorbing member 324 and/or the spring may be translatable within the handle 302. In some embodiments, the force absorbing member 324 and/or the spring may include a longitudinal axis oriented substantially parallel to a longitudinal axis of the handle 302 and/or the elongate shaft 310. In some embodiments, the force absorbing member 324 and/or the spring may be axially translatable within the handle 302. In some embodiments, the force absorbing member 324 and/or the spring may be rotatable within the handle 302. In some embodiments, the force absorbing member 324 and/or the spring may be translatable in a transverse direction with respect to the handle 302 and/or the longitudinal axis of the handle 302. Other configurations, including combinations thereof, are also contemplated. Some suitable, but non-limiting, examples of materials for the force absorbing member 324 and/or the spring, including but not limited to polymeric and/or metallic materials, are discussed below.

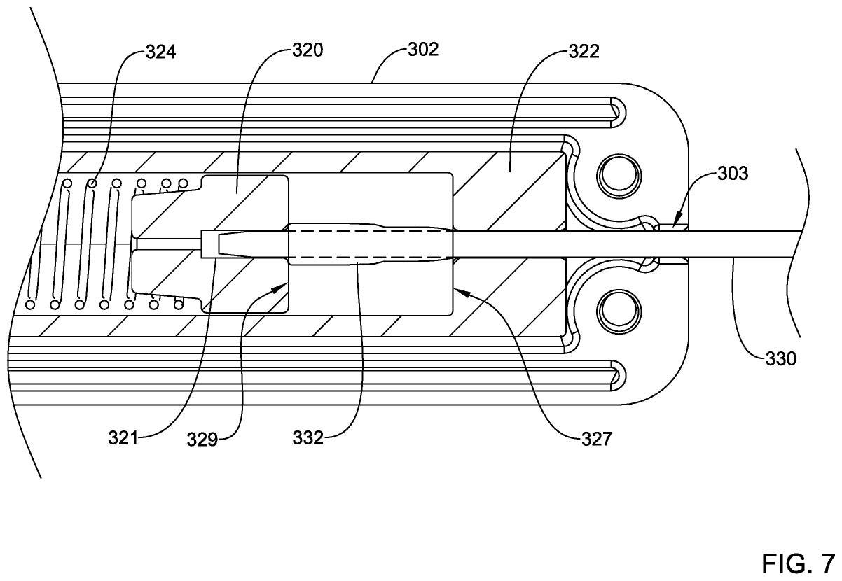

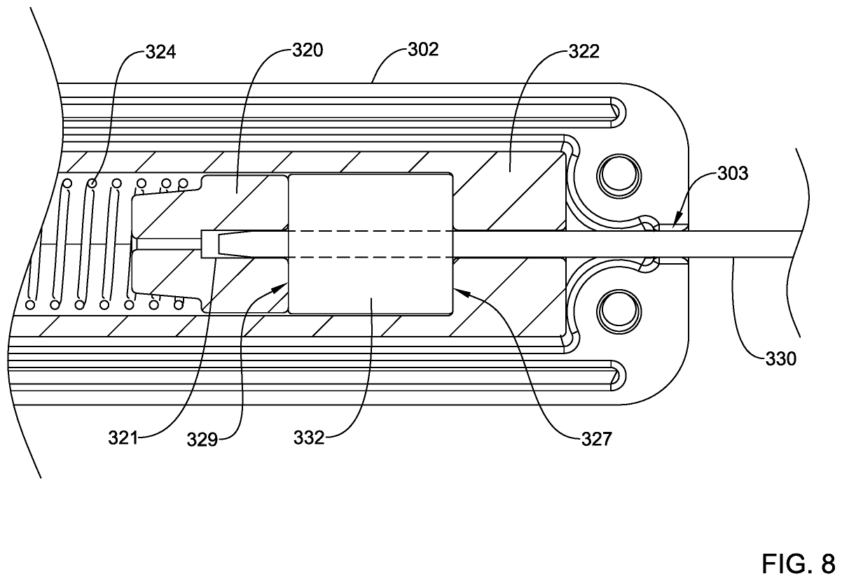

In some embodiments, the medical device 300 may include an inflatable balloon 332 disposed within the handle 302. The inflatable balloon 332 may be configured to shift between a deflated configuration (e.g., FIGS. 3, 7) and an inflated configuration (e.g., FIGS. 8, 14, 17, 19). In some embodiments, the medical device 300 may include a balloon shaft 330 fixedly attached to the inflatable balloon 332 and having an inflation lumen extending from the inflatable balloon 332 and/or the handle 302 to a regulator 370 disposed outside of the handle 302. The regulator 370 may be in fluid communication with a source of inflation fluid 372 and the balloon shaft 330. In one preferred example, the inflation fluid may be a biocompatible gas, such as air, oxygen, etc. In another example, the inflation fluid may be a biocompatible liquid, such as saline, etc. Other configurations and/or inflation fluids are also contemplated.

In some embodiments, the balloon shaft 330 may extend proximally of and/or from the handle 302 and engage with a longitudinal advancement-retraction mechanism 360, such as a bidirectional longitudinal advancement and retraction mechanism (e.g., a reel, a coil, etc.), disposed proximal of the handle 302. In some embodiments, the longitudinal advancement-retraction mechanism 360 may include a rotating element disposed within a longitudinal advancement-retraction mechanism housing, wherein the balloon shaft 330 is configured to wind or coil around the rotating element as the balloon shaft 330 is translated axially relative to the longitudinal advancement-retraction mechanism 360 and/or the handle 302. In some embodiments, the longitudinal advancement-retraction mechanism 360 may include a spring mechanism or other self-biasing element disposed within the longitudinal advancement-retraction mechanism housing that is configured to bias the rotating element to “reel up” the balloon shaft 330 as the balloon shaft 330 is translated proximally relative to the longitudinal advancement-retraction mechanism 360 and/or the handle 302, and is configured to permit the rotating element to “unreel” the balloon shaft 330 as the balloon shaft 330 is translated distally relative to the longitudinal advancement-retraction mechanism 360 and/or the handle 302.

In some embodiments, the medical device 300 may include a pressure sensor 374 in fluid communication with the balloon shaft 330 and/or the inflation lumen disposed therein. In some embodiments, the pressure sensor 374 may extend into and/or may be positioned within the balloon shaft 330 and/or the inflation lumen. In some embodiments, the pressure sensor 374 may be configured to measure and/or monitor an internal pressure within the inflatable balloon 332 via the balloon shaft 330 and/or the inflation lumen. In some embodiments, the pressure sensor 374 may be disposed directly within and/or in direct fluid communication with the inflatable balloon 332. In some embodiments, the medical device 300 may include a connection 375 to a pressure control 376. In some embodiments, the connection 375 may be a physical connection such as a wire. In some embodiments, the connection 375 may be a wireless connection. In some embodiments, the pressure control 376 may optionally be in electronic communication with a monitoring device 378, such as a display, a computer, etc.

In some embodiments, the monitoring device 378 may be configured to record and/or store values of the force applied to esophageal tissue by the distal nub 312 and/or the internal pressure within the inflatable balloon 332 sampled over time. In some embodiments, the pressure control 376 and/or monitoring device 378 may include and/or emit visual and/or audible alarms when the force exceeds a predetermined maximum force and/or when the force drops below a predetermined force threshold. In some embodiments, the pressure control 376 and/or monitoring device 378 may include and/or emit visual and/or audible alarms when the internal pressure exceeds a preset pressure limit and/or if the internal pressure drops below a predetermined pressure threshold. In some embodiments, the pressure control 376 and/or monitoring device 378 may include and/or may be in electronic communication with a computer, a microprocessor, a network, a cloud computing service, or other communications equipment.

In some embodiments, the inflatable balloon 332 may cooperate with the force absorbing member 324 to exert the force along the elongate shaft 310 to the distal end of the upper esophageal pouch 22. In at least some embodiments, the internal pressure within the inflatable balloon 332 increases as the force absorbing member 324 is compressed. Additional details will be discussed herein.

The medical device 300 and/or the handle 302 may include an adjustment mechanism configured to change the internal pressure within the inflatable balloon 332 and/or configured to change the force applied to the distal end of the upper esophageal pouch 22 by the elongate shaft 310 and the distal nub 312. In at least some embodiments, the adjustment mechanism may be configured to axially translate the force absorbing member 324 and/or the inflatable balloon 332 within the handle 100.

In some embodiments, the handle 302 and/or the adjustment mechanism may include a knob 306 configured to rotate relative to the handle 302. In some embodiments, the knob 306 may be configured to rotate around the longitudinal axis of the handle 302. In some embodiments, the knob 306 may be disposed proximate a proximal end of the handle 302. In some embodiments, the knob 306 may be disposed proximate a distal end of the handle 302. In some embodiments, the knob 306 may be disposed at a medial portion of the handle 302. In some embodiments, the knob 306 may extend transversely through the handle 302. Other positions are also contemplated. In some embodiments, the knob 306 may be fixed axially with respect to the handle 302. In some embodiments, the knob 306 is prevented from axial movement relative to the handle 302. In some alternative embodiments, the knob 306 may be configured to translate axially with respect to the handle 302 and may be non-rotatable with respect to the handle 302. Other configurations are also contemplated. Some suitable, but non-limiting, examples of materials for the knob 306, including but not limited to polymeric, composite, and/or metallic materials, are discussed below.

In some alternative embodiments, the adjustment mechanism may include a fluid and/or be adjusted or actuated by a fluid (e.g., water, saline, a biocompatible fluid, etc.). In some embodiments, the adjustment mechanism may include a gas and/or be adjusted or actuated by a gas (e.g., air, a biocompatible gas, etc.). In some embodiments, the adjustment mechanism may include a motorized adjustment and/or be adjusted or actuated by a motorized adjustment. Other configurations, including combinations thereof, are also contemplated.

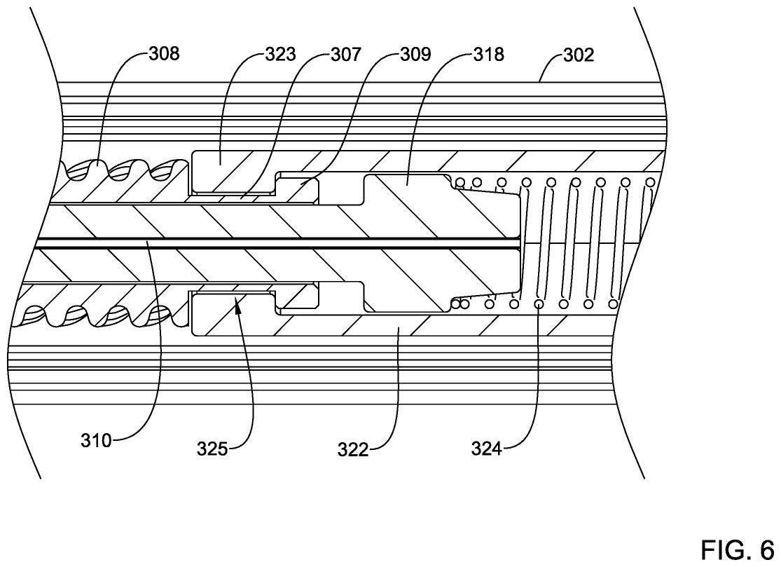

In some embodiments, the adjustment mechanism may include a threaded shaft 308 engaged with the knob 306 within the handle 302. In some embodiments, the threaded shaft 308 may extend proximally and/or distally from the knob 306 within the handle 302. In some embodiments, a first end of the threaded shaft 308 may engage a first end of a tubular retainer 322, as seen in greater detail in FIG. 6. In some embodiments, the threaded shaft 308 may include a flange 309 extending radially outward at the first end and a neck 307 extending from a threaded portion of the threaded shaft 308 to the flange 309, wherein an outer surface of the neck 307 is disposed radially inward of the flange 309. The first end of the tubular retainer 322 may include a lip 323 extending radially inward to an aperture 325. The neck 307 of the threaded shaft 308 may be disposed within the aperture 325 of the tubular retainer 322 such that the flange 309 of the threaded shaft 308 is disposed within the tubular retainer 322. In some embodiments, the threaded shaft 308 and the tubular retainer 322 may be axially inseparable (e.g., cannot be pulled apart in an axial direction).

Returning to FIG. 3, in some embodiments, the knob 306 may include internal threads configured to engage with external threads formed on and/or along an outer surface of the threaded shaft 308. The threaded shaft 308 may be configured to translate axially within the handle 302 in response to rotation of the knob 306. As such, actuation of the adjustment mechanism and/or rotation of the knob 306 relative to the handle 302 may translate the threaded shaft 308 and the tubular retainer 322 axially within the handle 302. For example, rotation of the knob 306 is a first rotational direction (e.g., clockwise) causes or results in axial movement of the threaded shaft 308 and the tubular retainer 322 in a distal direction relative to the handle 302 and/or the knob 306, and rotation of the knob 306 in a second rotation direction (e.g., counter-clockwise) opposite the first rotational direction causes or results in axial movement of the threaded shaft 308 and the tubular retainer 322 in a proximal direction relative to the handle 302 and/or the knob 306. In at least some embodiments, the first end of the threaded shaft 308 may be a proximal end of the threaded shaft 308 and the first end of the tubular retainer 322 may be a distal end of the tubular retainer 322. The force absorbing member 324 and/or the inflatable balloon 332 may be disposed within the tubular retainer 322. In some embodiments, actuation of the adjustment mechanism and/or rotation of the knob 306 relative to the handle 302 may translate the force absorbing member 324 and/or the inflatable balloon 332 axially within the handle 302.

In some embodiments, the elongate shaft 310 may extend axially through the knob 306 and/or may extend axially through a longitudinally oriented lumen formed in the threaded shaft 308. In some embodiments, the elongate shaft 310 may be movable with respect to the knob 306 and/or the threaded shaft 308. In some embodiments, the elongate shaft 310 may be axially translatable with respect to the knob 306 and/or the threaded shaft 308.

A proximal end of the elongate shaft 310 may be fixedly attached to a plunger 318. A distal portion of the plunger 318 may be slidably received within the longitudinally oriented lumen of the threaded shaft 308. The distal portion of the plunger 318 may have a first outer extent configured to cooperate with the longitudinally oriented lumen of the threaded shaft 308. A proximal portion of the plunger 318 may include a head configured to engage the force absorbing member 324, as shown in greater detail in FIG. 6. The head may be slidably disposed within the tubular retainer 322. The head may have a second outer extent greater than the first outer extent of the distal portion of the plunger 318. In some embodiments, the head may include a proximally facing flange configured to abut a distal end of the force absorbing member 324. In at least some embodiments, the distal end of the force absorbing member 324 may be secured to and/or fixedly attached to the head of the plunger 318 and/or to the proximally facing flange.

As seen generally in FIG. 3, and in greater detail in FIGS. 7-8, in some embodiments, a proximal end of the force absorbing member 324 may be configured to engage a proximal slider 320. The proximal slider 320 may be slidably disposed within the tubular retainer 322. The proximal slider 320 may be spaced apart from the plunger 318 by the force absorbing member 324. In some embodiments, the proximal slider 320 may include a distally facing flange configured to abut the proximal end of the force absorbing member 324. In at least some embodiments, the proximal end of the force absorbing member 324 may be secured to and/or fixedly attached to the proximal slider 320. In some embodiments, the proximal slider 320 may include a cavity 321 configured to receive a distal tip of the balloon shaft 330 extending distally from a proximal surface 329 of the proximal slider 320. It should be noted that to improve clarity, the balloon shaft 330 and the inflatable balloon 332 are not shown in section. In some embodiments, the balloon shaft 330 may include a lumen extending therethrough and in communication with a lumen formed through the proximal slider 320 to permit passage through the medical device 300 of a wire or other element, for example.

The inflatable balloon 332 may be disposed within the tubular retainer 322. The inflatable balloon 332 may be disposed between the proximal surface 329 of the proximal slider 320 and a distally facing wall 327 at a second end (e.g., a proximal end) of the tubular retainer 322. The inflatable balloon 332 is shown in FIG. 7 in a deflated configuration and in FIG. 8 in an inflated configuration. The handle 302 may include a proximal aperture 303 configured to slidably receive the balloon shaft 330 therethrough. In some embodiments, the proximal aperture 303 may be disposed at a proximalmost end of the handle 302. A distal portion or a distal surface of the inflatable balloon 332 may be configured to engage with and/or contact the proximal surface 329 of the proximal slider 320 in the inflated configuration. A proximal portion or a proximal surface of the inflatable balloon 332 may be configured to engage with and/or contact the distally facing wall 327 at the second end of the tubular retainer 322 in the inflated configuration. The proximal slider 320 may be biased distally by the inflatable balloon 332 in the inflated configuration. The proximal slider 320 may be biased proximally by the force absorbing member 324. Some suitable, but non-limiting, examples of materials for the threaded shaft 308, the tubular retainer 322, the plunger 318, and/or the proximal slider 320, including but not limited to polymeric, composite, and/or metallic materials, are discussed below.

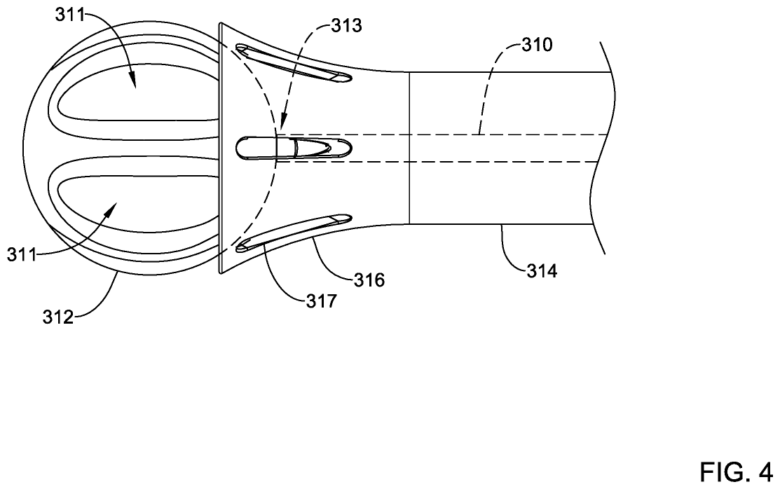

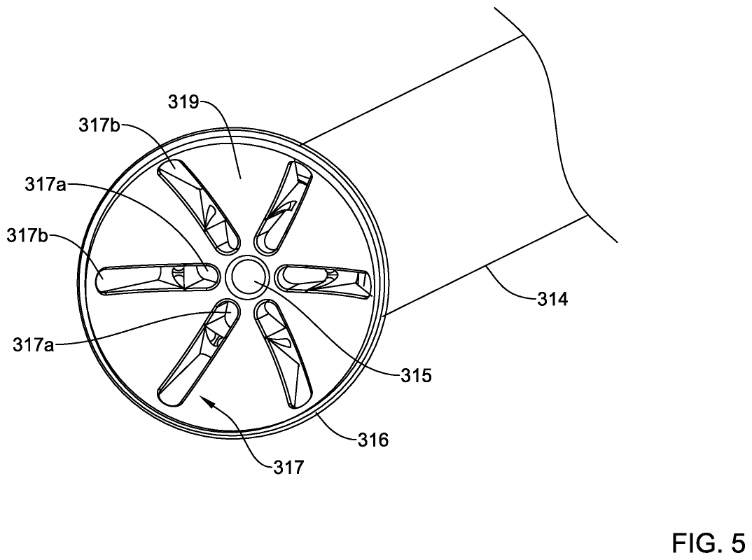

Turning now to FIGS. 4-5, selected aspects of the distal end of the elongate tubular member 314, the distal end of the elongate shaft 310, and/or the distal nub 312 are illustrated. In FIG. 5, the elongate shaft 310 and the distal nub 312 are removed for clarity.

The elongate shaft 310 may be axially movable within and/or may be slidably disposed within a lumen of the elongate tubular member 314. As discussed herein, prior to preparing the medical device 300 for use, the distal nub 312 may be detached from the elongate shaft 310. In some embodiments, the plurality of distal nubs is detached from the elongate shaft 310 prior to preparing the medical device 300 for use. After assessing characteristics of the distal end of the upper esophageal pouch 22, the distal nub 312 (or one of the plurality of distal nubs) may be attached to the distal end of the elongate shaft 310 at and/or using a non-releasable connector 313. In some embodiments, the distal end of the elongate shaft 310 may include a female portion of the non-releasable connector 313 and the distal nub 312 may include a male portion of the non-releasable connector 313 configured to non-releasably secure the distal nub 312 to the elongate shaft 310. In some embodiments, the distal end of the elongate shaft 310 may include a male portion of the non-releasable connector 313 and the distal nub 312 may include a female portion of the non-releasable connector 313 configured to non-releasably secure the distal nub 312 to the elongate shaft 310. Other configurations are also contemplated.

The elongate tubular member 314 may include a distal basket 316 configured to receive at least a portion of the distal nub 312. In some instances, the distal basket 316 may be a conical structure secured to and flaring outward from the distal end of the elongate tubular member 314. In some embodiments, a distal facing surface 319 (e.g., FIG. 5) of the distal basket 316 may be configured to engage and/or conform to a proximal surface of the distal nub 312. The distal basket 316 may include a centering ring 315 disposed at a central portion of the distal basket 316. The centering ring 315 may be configured to slidably receive the elongate shaft 310. In at least some embodiments, the centering ring 315 may be disposed and/or positioned coaxially with a central longitudinal axis of the elongate tubular member 314 and/or the elongate shaft 310.

The distal basket 316 may include a plurality of apertures 317 formed therein. The plurality of apertures 317 may include a plurality of first apertures 317a extending between the distal facing surface 319 of the distal basket 316 and the lumen of the elongate tubular member 314. The plurality of apertures 317 may include a plurality of second apertures 317b extending between the distal facing surface 319 of the distal basket 316 and a proximal facing surface of the distal basket 316. In at least some embodiments, the plurality of first apertures 317a and the plurality of second apertures 317b may be in fluid communication with each other within the distal basket 316.

The distal basket 316 and/or the plurality of apertures 317 may be configured such that suction of mucous and/or saliva from the upper esophageal pouch 22 is permitted regardless of a position of the distal nub 312 relative to the distal basket 316. For example, the distal basket 316 and/or the plurality of apertures 317 may permit suction of mucous and/or saliva from the upper esophageal pouch 22 even if the distal nub 312 is disposed against and/or is in conforming engagement with the distal facing surface 319 of the distal basket 316.

In some embodiments, the distal nub 312 may include one or more, or a plurality of flutes arranged around the distal nub 312. For example, the distal nub 312 may include at least one groove 311 (e.g., FIG. 4) formed in an outer surface of the distal nub 312. In some embodiments, the at least one groove 311 may include at least one channel, at least one elongated recess, etc. The at least one groove 311 may permit suction of mucous and/or saliva around the distal nub 312 if and/or when the distal nub 312 is engaged with and/or at least partially received within the distal basket 316. In some embodiments, the at least one groove 311 may be in fluid communication with the plurality of apertures 317 and/or the plurality of first apertures 317a when the distal nub 312 is engaged with and/or at least partially received within the distal basket 316. The at least one groove 311 may permit suction of mucous and/or saliva from the upper esophageal pouch 22 around the distal nub 312 even if the distal nub 312 is disposed against and/or is in conforming engagement with the distal facing surface 319 of the distal basket 316.

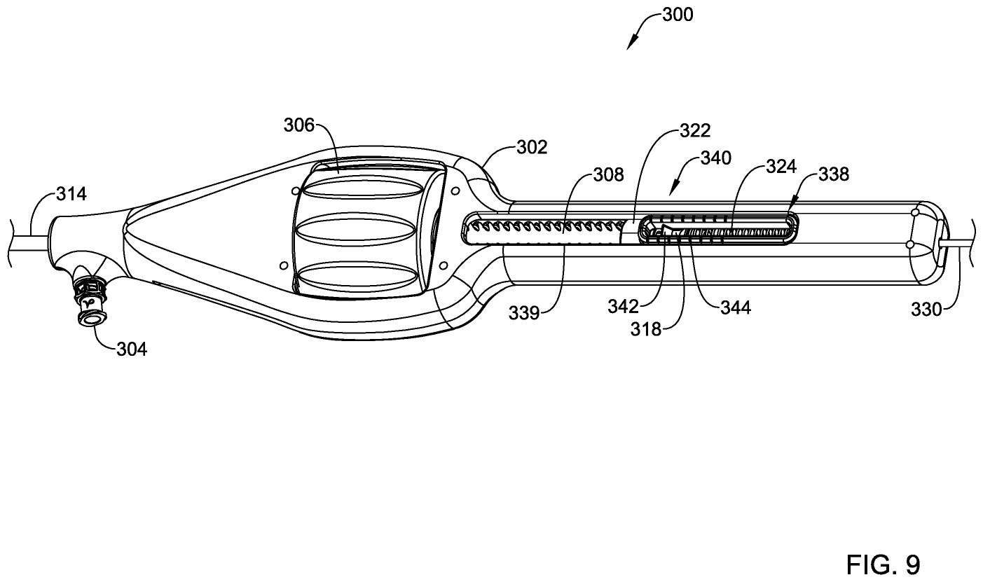

FIG. 9 illustrates selected aspects of the medical device 300 as seen from an opposite side of the handle 302 compared to FIG. 2 (e.g., the handle 302 has been flipped over). The handle 302 may include a cutout 339 through a side wall of the handle 302. In some embodiments, one or more components of the medical device 300 disposed within the handle 302 may be seen through the cutout 339. For example, in FIG. 9, the threaded shaft 308 and the tubular retainer 322 may be seen through the cutout 339. In some embodiments, the cutout 339 may be covered and/or shielded with a cover (not shown). In some embodiments, cover may be transparent, partially transparent, or opaque. In some embodiments, the cover may seal the cutout 339 to protect the components of the medical device 300 from contamination. Other configurations are also contemplated.

As seen in FIG. 9, in some embodiments, the tubular retainer 322 may include a radially projecting rim 338 extending into the cutout 339. The radially projecting rim 338 may have an elongated shape extending in a longitudinal direction. In at least some embodiments, the radially projecting rim 338 may be configured to slide axially within the cutout 339 as the tubular retainer 322 is translated axially within the handle 302. The radially projecting rim 338 may define a window into the handle 302 and/or the tubular retainer 322. In some embodiments, at least a portion of the plunger 318, at least a portion of the force absorbing member 324, and/or at least a portion of the proximal slider 320 may be visible within and/or through the window defined by the radially projecting rim 338.

In some embodiments, the medical device 300 and/or the handle 302 may include a position indicator 340 configured to communicate an axial position of the elongate shaft 310 relative to the handle 302. In some embodiments, the position indicator 340 may include a plurality of markings 344 on the radially projecting rim 338 of the tubular retainer 322. In some embodiments, the plurality of markings 344 may include a plurality of notches formed in the radially projecting rim 338 and/or the handle 302. In some embodiments, the plurality of markings 344 may include marks, shapes, patterns, etc. printed on the radially projecting rim 338 and/or the handle 302. Other configurations are also contemplated. In some embodiments, the position indicator 340 may include a tab 342 extending radially outward from the plunger 318 into the window defined by the radially projecting rim 338. As will be discussed herein, at an early stage of a procedure, the tab 342 may be disposed at a more proximal position within the window. As the upper esophageal pouch 22 grows and/or stretches, the tab 342 may translate distally within the window as the elongate shaft 310 translates distally relative to the handle 302 and/or as the force absorbing member 324 elongates. The relative position of the tab 342 with respect to the plurality of markings 344 may provide a visual indication of the axial position of the elongate shaft 310 and/or of a change in the axial position of the elongate shaft 310. Other types of position indicators, including but not limited to electronic devices and/or sensors, are also contemplated.

In some embodiments, as the medical device 300 is used and/or adjusted during a procedure, the tubular retainer 322 may translate axially within the handle 302. As the tubular retainer 322 translates axially within the handle 302, the radially projecting rim 338 and the window defined by the radially projecting rim 338 may also translate axially within the handle 302 and/or within the cutout 339. As such, as adjustments are made, even if the handle 302 is not moved relative to the patient, the position indicator 340 may follow the adjustments and thus continue to provide the visual indicator of the relative position of the elongate shaft 310.

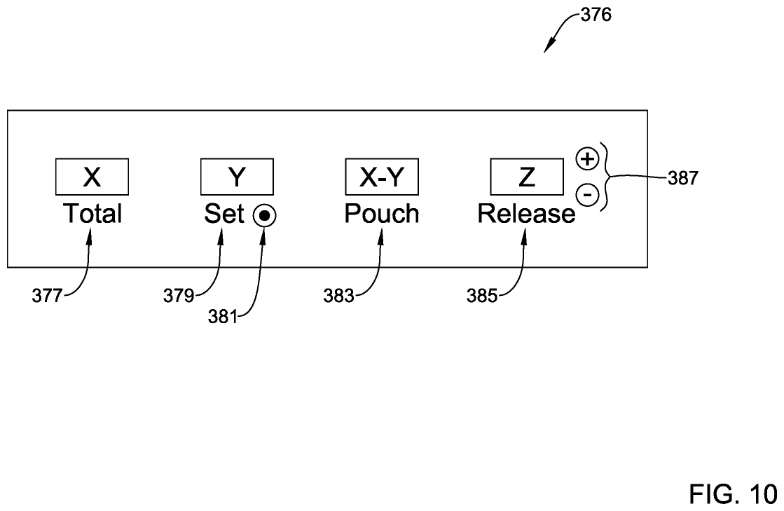

FIG. 10 illustrates an example configuration of the pressure control 376. The pressure control 376 may be configured to set, monitor, display, and/or react to the internal pressure within the inflatable balloon 332 and/or a pressure and/or force being exerted and/or applied along the elongate shaft 310 to the distal end of the upper esophageal pouch 22. As shown in FIG. 2, the pressure control 376 may be operably connected to the pressure sensor 374 by the connection 375. In at least some embodiments, the pressure control 376 may display and/or monitor the pressure in units of mmHg (e.g., millimeters of mercury). Other units and/or measurements are also contemplated. In some embodiments, the pressure control 376 may include known and/or predetermined data related to the force absorbing member 324 stored therein, built into the pressure control 376, and/or referenced by one or more algorithms used by the pressure control 376. In some embodiments, the pressure control 376 may include a pressure release valve configured to release pressure from within the inflatable balloon 332 and the inflation lumen when a preset pressure limit is exceeded. In some embodiments, the pressure control 376 may be operably connected to the regulator 370 to control the flow of inflation fluid into and/or out of the balloon shaft 330, the inflation lumen, and/or the inflatable balloon 332.

In some embodiments, the pressure control 376 may include a Total Pressure display 377, shown as “X” (e.g., X mmHg), a Set Pressure display 379, shown as “Y” (e.g., Y mmHg), a Pouch Pressure display 383, which is calculated and shown as “X−Y” (e.g., the Total Pressure X minus the Set Pressure Y), and a Release Pressure display 385, shown as “Z” (e.g., Z mmHg). The Total Pressure corresponds to the internal pressure within the inflatable balloon 332 and the inflation lumen. During use, as the force absorbing member 324 is compressed or elongated, the internal pressure within the inflatable balloon 332 and the inflation lumen may increase or decrease, respectively. The Set Pressure corresponds to the internal pressure within the inflatable balloon 332 and the inflation lumen prior to positioning the distal nub 312 against the distal end of the upper esophageal pouch 22. The pressure control 376 may include a set pressure button 381 adjacent the Set Pressure display 379, wherein the set pressure button 381 may be used to lock the internal pressure within the inflatable balloon 332 and the inflation lumen at an initial pressure or starting point, which is shown as the Set Pressure. The Pouch Pressure reflects an amount of pressure being exerted against the distal end of the upper esophageal pouch 22 by the distal nub 312 and may be determined from any change in the internal pressure within the inflatable balloon 332 and the inflation lumen. The pressure control 376 may include release pressure adjustment buttons 387 disposed adjacent the Release Pressure display 385. The release pressure adjustment buttons 387 may be used to adjust and/or set the preset pressure limit for the Total Pressure, the Pouch Pressure, and/or the internal pressure within the inflatable balloon 332. In one example, if the Total Pressure (e.g., X mmHg) is greater than or equal to the Release Pressure (e.g., Z mmHg), the pressure control 376 and/or the regulator 370 may release some or all of the internal pressure within the inflatable balloon 332 and the inflation lumen, thereby deflating the inflatable balloon 332 and reducing and/or removing the pressure and/or force being exerted against the distal end of the upper esophageal pouch 22. In another example, if the Pouch Pressure (e.g., X−Y mmHg) is greater than or equal to the Release Pressure (e.g., Z mmHg), the pressure control 376 and/or the regulator 370 may release some or all of the internal pressure within the inflatable balloon 332 and the inflation lumen, thereby deflating the inflatable balloon 332 and reducing and/or removing the pressure and/or force being exerted against the distal end of the upper esophageal pouch 22. Other configurations are also contemplated.

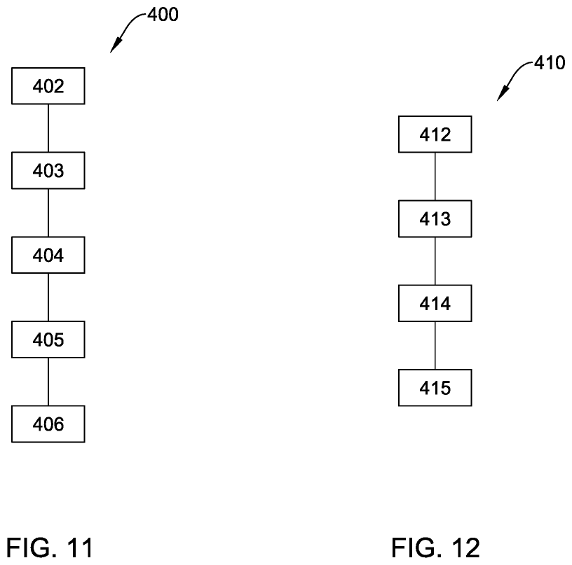

FIG. 11 is a block diagram illustrating selected aspects of a pre-placement procedure 400 for the medical device 300. The pre-placement procedure 400 includes actions and/or activities that are performed prior to inserting the distal nub 312 into the patient 10.

At step 402, the pre-placement procedure 400 may include attaching and/or connecting the fluid management system 305 to the side port 304. In some embodiments, at step 402, the pre-placement procedure 400 may include attaching and/or connecting an irrigation lumen and/or a suction lumen to the side port 304. In some embodiments, at step 402, the pre-placement procedure 400 may include inserting an irrigation tube and/or a suction tube into the side port 304.

At step 403, the pre-placement procedure 400 may include attaching the distal nub 312 to the distal end of the elongate shaft 310. In some embodiments, at step 403, the pre-placement procedure 400 may include selecting one of the plurality of distal nubs and attaching the selected distal nub to the distal end of the elongate shaft 310. In some embodiments, at step 403 (or at a step prior to step 403), the pre-placement procedure 400 may include assessing characteristics of the distal end of the upper esophageal pouch 22 used to select one of the plurality of distal nubs.

At step 404, the pre-placement procedure 400 may include inflating the internal pressure within the inflatable balloon 332 and the inflation lumen to the initial pressure. The initial pressure may be any desired amount. In one example, the initial pressure may be 10 mmHg. Other values and/or configurations are also contemplated. For the purpose of illustration only, using the above example, at this stage in the pre-placement procedure 400, the Total Pressure display 377 and the Set Pressure display 379 may each show 10 mmHg (e.g., X and Y are each 10 mmHg), and the Pouch Pressure display 383 may show 0 mmHg (e.g., X−Y is 0 mmHg).

At step 405, the pre-placement procedure 400 may include locking the Set Pressure (Y) using the set pressure button 381. In at least some embodiments, the Set Pressure (Y) may be locked at the initial pressure. When the Set Pressure (Y) has been locked, the regulator 370 and/or any valve permitting inflation fluid to flow into the inflation lumen and the inflatable balloon 332 may be closed. As such, the internal pressure within the inflatable balloon 332 and the inflation lumen, when there is no outside force being exerted upon the inflatable balloon 332, will be locked and/or maintained at the Set Pressure (Y).

At step 406, the pre-placement procedure 400 may include setting the Release Pressure (Z) (e.g., the preset pressure limit) using the release pressure adjustment buttons 387. The Release Pressure (Z) may be any desired amount but may be based upon limits known to prevent tissue damage and/or injury to the patient 10 at the distal end of the upper esophageal pouch 22. The Release Pressure display 385 may correspond to the preset pressure limit at which the pressure control 376 and/or the regulator 370 may be configured to deflate the inflatable balloon 332 and the inflation lumen. In one example, the preset pressure limit (e.g., the Release Pressure (Z)) may be 50 mmHg. Other values and/or configurations are also contemplated. For the purpose of illustration only, using the above examples, at this stage in the pre-placement procedure 400, the Total Pressure display 377 and the Set Pressure display 379 may each show 10 mmHg (e.g., X and Y are each 10 mmHg), the Pouch Pressure display 383 may show 0 mmHg (e.g., X−Y is 0 mmHg), and the Release Pressure display 385 may show 50 mmHg (e.g., Z is 50 mmHg). At this stage in the pre-placement procedure 400, the distal nub 312 remains outside of the patient 10 and/or out of contact with the distal end of the upper esophageal pouch 22.

FIG. 12 is a block diagram illustrating selected aspects of a placement procedure 410 for the medical device 300. The placement procedure 410 includes actions and/or activities that are performed during insertion of the distal nub 312 into the patient 10. At step 412, the placement procedure 410 may include activating the fluid management system 305. In some embodiments, the placement procedure 410 may include activating any irrigation and/or suction attached to, connected to, and/or in communication with the side port 304 of the medical device 300. In one example, at step 412, the placement procedure 410 may include activating suction (e.g., activating the source of suction or the vacuum source) attached to, connected to, and/or in communication with the side port 304 of the medical device 300.

At step 413, the placement procedure 410 may include inserting the distal nub 312 into the esophagus 20 and/or the upper esophageal pouch 22. In some embodiments, at step 413, the placement procedure 410 may include inserting and/or advancing the distal nub 312 into the upper esophageal pouch 22 until the distal nub 312 is in contact with the distal end of the upper esophageal pouch 22. In some embodiments, at step 413, the placement procedure 410 may include inserting and/or advancing the distal nub 312 into the upper esophageal pouch 22 until the user “feels” the distal nub 312 is in contact with the distal end of the upper esophageal pouch 22. In some embodiments, at step 413, when the distal nub 312 contacts the distal end of the upper esophageal pouch 22, attempts at further advancement and/or insertion of the distal nub 312 may result in the Total Pressure display 377 showing an increase in Total Pressure (X) to a value greater than the Set Pressure (Y) and the Pouch Pressure display 383 showing an increase in Pouch Pressure (X−Y) to a value greater than zero.

At step 414, the placement procedure 410 may include withdrawing the handle 302 and/or the distal nub 312 proximally until the Pouch Pressure display 383 shows 0 mmHg (e.g., X−Y is 0 mmHg). In some embodiments, at step 414, the placement procedure 410 may include withdrawing the handle 302 and/or the distal nub 312 proximally until the Total Pressure display 377 shows the Set Pressure (Y). For the purpose of illustration only, using the above examples, at this stage of the placement procedure 410, the Total Pressure display 377 and the Set Pressure display 379 may each show 10 mmHg (e.g., X and Y are both 10 mmHg), and the Pouch Pressure display 383 may show 0 mmHg (e.g., X−Y is 0 mmHg). When the Pouch Pressure display 383 shows 0 mmHg, movement of the handle 302 and/or the distal nub 312 is immediately terminated and the handle 302 and/or the distal nub 312 is held in a fixed position relative to the patient 10.

At step 415, the placement procedure 410 may include securing the medical device 300 relative to the fixation element 380 and/or the patient 10. In some embodiments, at step 415, the placement procedure 410 may include securing the elongate tubular member 314 relative to the fixation element 380 and/or the patient 10. In some embodiments, at step 415, the placement procedure 410 may include securing the medical device 300 to the fixation element 380 and/or the patient 10. In some embodiments, at step 415, the placement procedure 410 may include securing the elongate tubular member 314 to the fixation element 380 and/or the patient 10. In some embodiments, at step 415, the placement procedure 410 may include securing the handle 302 (and/or other elements of the medical device 300 disposed proximal of the elongate tubular member 314) to an adjacent table or to the crib in which the patient 10 lies. Other configurations are also contemplated.

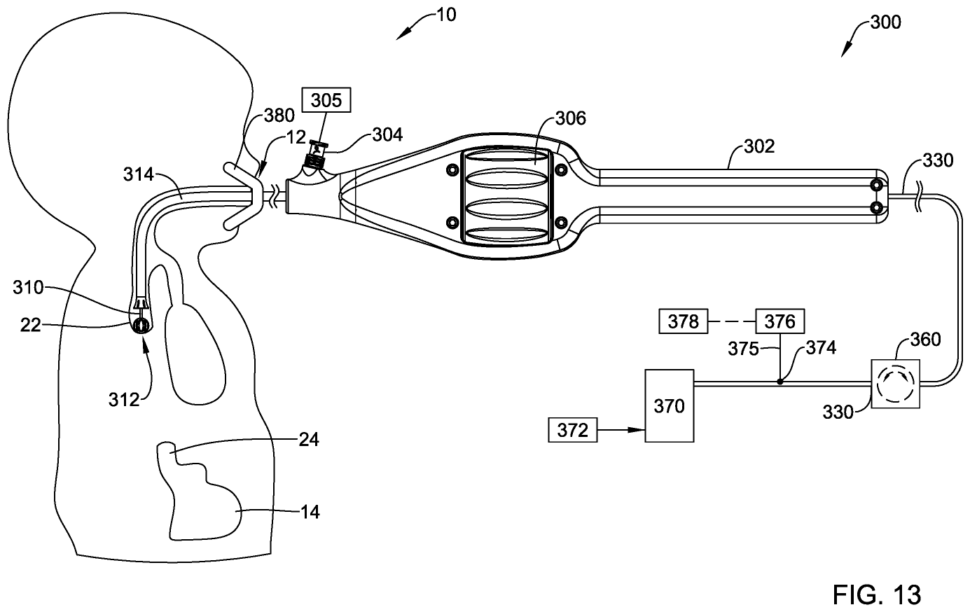

At and/or after completion of the placement procedure 410, the medical device 300 and/or the handle 302 may be fixed in place relative to the patient 10 with the distal nub 312 in contact with, but without exerting pressure on, the distal end of the upper esophageal pouch 22, as shown in FIG. 13. For the purpose of illustration only, using the above examples, at and/or after completion of the placement procedure 410, the Total Pressure display 377 and the Set Pressure display 379 may each show 10 mmHg (e.g., X and Y are both 10 mmHg), the Pouch Pressure display 383 may show 0 mmHg (e.g., X−Y is 0 mmHg), and the Release Pressure display 385 may show 50 mmHg (e.g., Z is 50 mmHg).

A method of treating esophageal atresia may include preparing the medical device 300 for treating esophageal atresia, as described herein. In some embodiments, the method and/or preparing the medical device 300 may include inflating the inflatable balloon 332 to an internal pressure, wherein the internal pressure is the initial pressure, as described herein. In some embodiments, the method may include positioning the distal nub 312 of the elongate shaft 310 and/or the medical device 300 against the distal end of the upper esophageal pouch 22, as seen in FIG. 13. In some embodiments, the method may include fixing the medical device 300 in place relative to the distal end of the upper esophageal pouch 22 and/or relative to the patient 10.

In some embodiments, the fixation element 380 may be secured to the face of the patient 10, such as with adhesive tape. In some embodiments, the fixation element 380 may include a headgear or a mouthpiece secured to the head of the patient 10. In some embodiments, the fixation element 380 may include a mask or other structure configured to at least partially surround and/or encompass the head of the patient 10. In some embodiments, the medical device 300 and/or the elongate tubular member 314 may be secured to the patient 10 and/or may be secured relative to the patient 10, the mouth 12, and/or the distal end of the upper esophageal pouch 22 by securing the medical device 300 and/or the elongate tubular member 314 to another medical device or product such as a NeoBar® from Neotech Products. Other suitable medical devices and/or products are also contemplated.

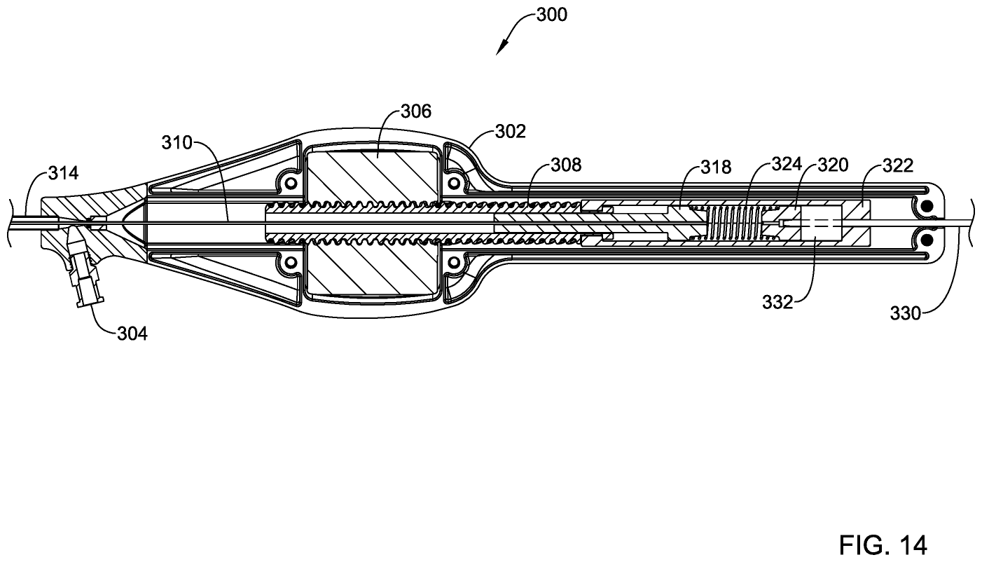

In some embodiments, the method may include actuating the adjustment mechanism and/or rotating the knob 306 relative to the handle 302 to change the internal pressure within the inflatable balloon 332 and the inflation lumen to a treatment pressure and/or to adjust the force applied to the distal end of the upper esophageal pouch 22 such that the Total Pressure (X) and/or the Pouch Pressure (X−Y) is at a desired value for treatment. Actuating the adjustment mechanism and/or rotating the knob 306 relative to the handle 302 may include translating the threaded shaft 308 and the tubular retainer 322 axially within the handle 302, as seen in FIG. 14. Since the distal nub 312 is already in contact with the distal end of the upper esophageal pouch 22, the elongate shaft 310 and the plunger 318 will remain in a generally constant position (e.g., will not translate axially) within and/or relative to the handle 302.

Axial translation of the tubular retainer 322 may cause the inflatable balloon 332, in the inflated configuration, to translate the proximal slider 320 axially toward the plunger 318. For example, distal axial translation of the tubular retainer 322 may cause the inflatable balloon 332, in the inflated configuration, to translate the proximal slider 320 distally toward the plunger 318. Axial translation of the proximal slider 320 toward the plunger 318 may axially compress the force absorbing member 324 between the proximal slider 320 and the plunger 318. The force absorbing member 324 may be biased to return to its uncompressed configuration. Axial compression of the force absorbing member 324 may cause the spring bias of the force absorbing member 324 to “push” the proximal slider 320 against the inflatable balloon 332, thereby squeezing the inflatable balloon 332 between the proximal slider 320 and the distally facing wall 327 of the tubular retainer 322. Accordingly, the internal pressure within the inflatable balloon 332 and the inflation lumen may increase as the force absorbing member 324 is compressed. The internal pressure may be monitored and/or displayed by the pressure control 376, as the Total Pressure (X) shown in the Total Pressure display 377. Similarly, the Pouch Pressure (X−Y) may be monitored and/or displaced by the pressure control 376. As such, actuating the adjustment mechanism and/or rotating the knob 306 relative to the handle 302 may produce and/or result in an increase in the internal pressure that may correspond to the amount of pressure and/or force being exerted along the elongate shaft 310 to apply the force to the distal end of the upper esophageal pouch 22 (e.g., the Pouch Pressure (X−Y)). This may be seen on the pressure control 376 in the Pouch Pressure display 383.

In some embodiments, the method may include actuating the adjustment mechanism and/or rotating the knob 306 relative to the handle 302 until the internal pressure within the inflatable balloon 332 and the inflation lumen (e.g., the Total Pressure (X) and/or the corresponding Pouch Pressure (X−Y)) is changed to a desired value. For the purpose of illustration only, using the above examples, after actuating the adjustment mechanism and/or rotating the knob 306 relative to the handle 302, the Total Pressure display 377 may show 40 mmHg (e.g., X is 40 mmHg) and the Set Pressure display 379 may show 10 mmHg (e.g., Y is 10 mmHg), the Pouch Pressure display 383 may show 30 mmHg (e.g., X−Y is 30 mmHg), and the Release Pressure display 385 may show 50 mmHg (e.g., Z is 50 mmHg).

In some embodiments, the method may include monitoring the internal pressure (e.g., the Total Pressure (X)) within the inflatable balloon 332 and the inflation lumen. In some embodiments, the method may include monitoring the Pouch Pressure (X−Y). In some embodiments, the method may include increasing or decreasing the Release Pressure (Z) using the release pressure adjustment buttons 387, if desired.

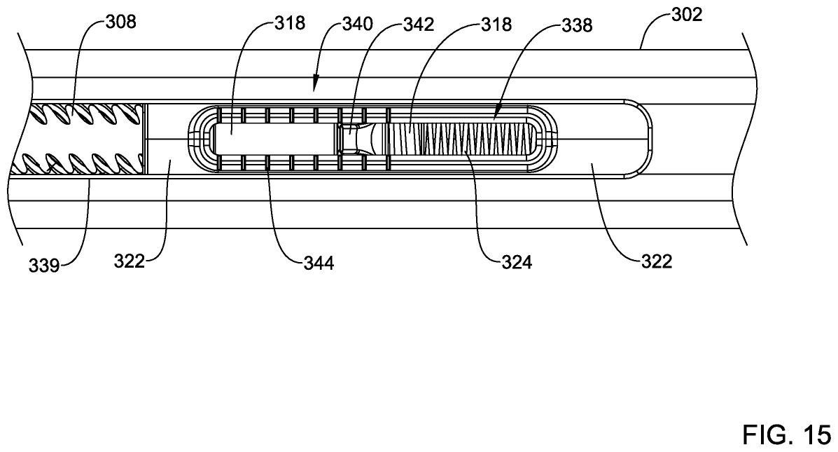

FIG. 15 illustrates the relative positioning of selected elements of the medical device 300 of FIG. 14, after actuating the adjustment mechanism and/or rotating the knob 306 relative to the handle 302 to translate the threaded shaft 308 and the tubular retainer 322 axially within the handle 302. As shown in FIG. 15, the position indicator 340 has shifted within the cutout 339 along with the radially projecting rim 338 and/or the tubular retainer 322. The tab 342 extending radially outward from the plunger 318 was maintained in a generally constant position by the engagement of the distal nub 312 with the distal end of the upper esophageal pouch 22, as described herein. The position of the tab 342 relative to the plurality of markings 344 may be noted and/or referenced during the procedure.

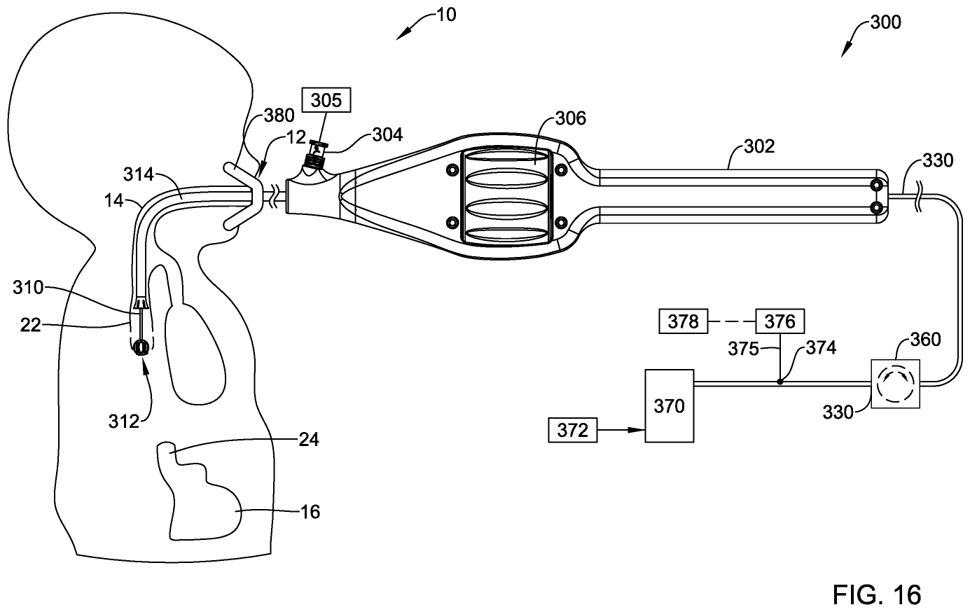

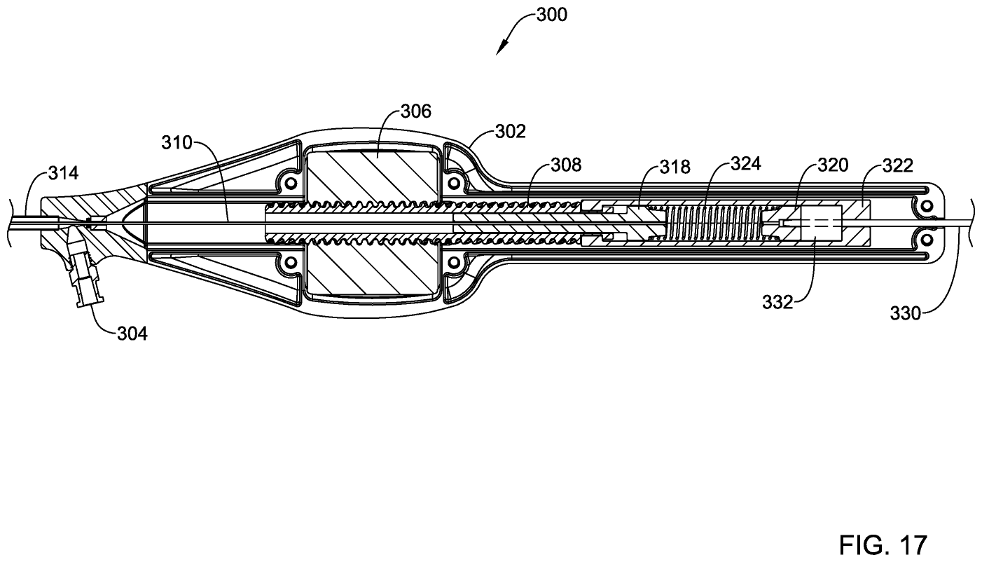

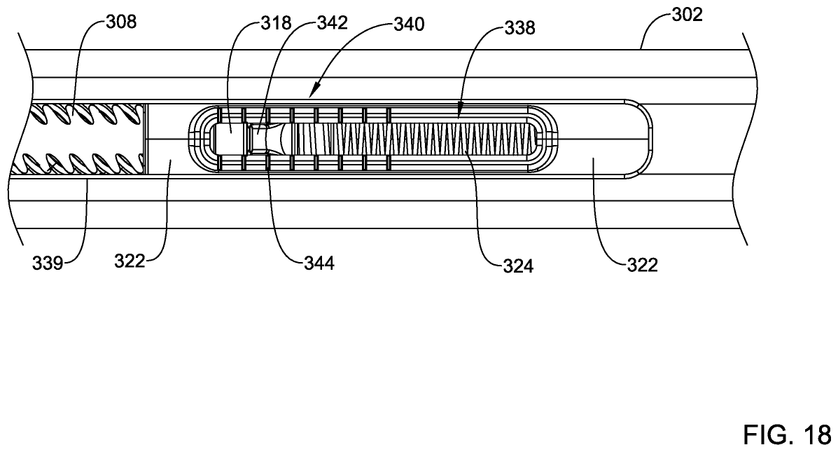

Over time, the distal end of the upper esophageal pouch 22 will grow and/or stretch as the distal end of the upper esophageal pouch 22 is subjected to the force exerted along the elongate shaft 310, as shown in FIG. 16. As the distal end of the upper esophageal pouch 22 grows and/or stretches, the plunger 318 may be biased away from the proximal slider 320 by the force absorbing member 324, which may slowly axially expand, decompress, and/or resilient expand, as seen in FIG. 17, while maintaining the distal nub 312 in contact with the distal end of the upper esophageal pouch 22, as seen in FIG. 16. Accordingly, the plunger 318 may be translated axially within and/or relative to the handle 302 and the elongate shaft 310 may similarly be translated axially relative to the handle 302 to maintain the distal nub 312 in contact with the distal end of the upper esophageal pouch 22. As the plunger 318 translates axially within the handle 302, the position indicator 340 may reflect the change(s), as seen in FIG. 18, wherein the tab 342 has translated within the window defined by the radially projecting rim 338 relative to the plurality of markings 344. The change in position of the tab 342 of the position indicator 340 (e.g., comparing FIG. 15 to FIG. 18) may be used to determine the amount the upper esophageal pouch 22 has grown or stretched over a period of time and/or the rate of elongation of the upper esophageal pouch 22.

As shown via arrows in FIG. 19, the force absorbing member 324 may be configured to permit the elongate shaft 310 and/or the plunger 318 to translate axially relative to the handle 302. In some embodiments, the force absorbing member 324 may be configured to permit the elongate shaft 310, the distal nub 312, and/or the plunger 318 to translate axially relative to the handle 302 in response to patient movements while maintaining the distal nub 312 against and/or in contact with the distal end of the upper esophageal pouch 22. Accordingly, as the patient 10 moves due to respiration, coughing, sneezing, crying, etc. the force absorbing member 324 may permit corresponding movement of the elongate shaft 310 and/or the distal nub 312 in an oscillatory manner (e.g., back and forth in an axial direction), thereby reducing the potential and/or opportunity for rupture for the upper esophageal pouch 22. In some embodiments, permitting axial oscillatory movement of the elongate shaft 310 and/or the distal nub 312 relative to the handle 302, and thus the distal end of the upper esophageal pouch 22, may also create and/or permit some occasional variation in the force applied to the distal end of the upper esophageal pouch 22 by the force absorbing member 324. Thus, the elongate shaft 310 and/or the distal nub 312 may cyclically move longitudinally or axially relative to the elongate tubular member 314 and/or handle 302 by cyclically absorbing some longitudinal movement of the elongate shaft 310 and/or the distal nub 312 with the force absorbing member 324. Such variation may permit and/or encourage reperfusion of the tissue at the distal end of the upper esophageal pouch 22, thereby reducing tissue morbidity and/or necrosis while providing sufficient force to induce growth and/or stretch of the distal end of the upper esophageal pouch 22 over time.

In some embodiments, as the threaded shaft 308, the tubular retainer 322, and the inflatable balloon 332 translate axially relative to the handle 302, the balloon shaft 330 may be taken up by and/or coiled by the longitudinal advancement-retraction mechanism 360 and/or released from and/or uncoiled from the longitudinal advancement-retraction mechanism 360, thereby preventing kinking of, damage to, etc. the balloon shaft 330 and/or any wire, fiber optic, etc. disposed therein. The longitudinal advancement-retraction mechanism 360 may also permit less of the balloon shaft 330 to be hanging freely outside of the handle 302, where the balloon shaft 330 may become tangled with other devices and/or equipment, etc.

In some embodiments, the method may include monitoring the internal pressure within the inflatable balloon 332 and the inflation lumen (e.g., Total Pressure (X)) over time. In some embodiments, the method may include monitoring the Pouch Pressure (X−Y) over time. In some embodiments, the method may include monitoring the force applied to the distal end of the upper esophageal pouch 22 over time. In some embodiments, the method may include monitoring axial translation of the elongate shaft 310 over time via the position indicator 340 or other means. In some embodiments, the method may include actuating the adjustment mechanism and/or rotating the knob 306, as seen in FIGS. 14 and 15, to increase the internal pressure within the inflatable balloon 332 and the inflation lumen when the internal pressure drops below a predetermined pressure threshold or when axial translation of the elongate shaft 110 reaches a predetermined translation threshold. In some embodiments, the method may include actuating the adjustment mechanism and/or rotating the knob 306, as seen in FIGS. 14 and 15, to increase the force applied to the distal end of the upper esophageal pouch 22 when the force drops below the predetermined force threshold or when axial translation of the elongate shaft 110 reaches a predetermined translation threshold.

In some embodiments, the predetermined pressure threshold may be the Set Pressure (Y) and/or the initial pressure. In some embodiments, the predetermined pressure threshold may be about 5% greater than the Set Pressure (Y) and/or the initial pressure. In some embodiments, the predetermined pressure threshold may be about 10% greater than the Set Pressure (Y) and/or the initial pressure. In some embodiments, the predetermined pressure threshold may be about 15% greater than the Set Pressure (Y) and/or the initial pressure. Other configurations and/or values are also contemplated.

In some embodiments, the predetermined force threshold may be 0.5 pounds of force. In some embodiments, the predetermined force threshold may be 0.4 pounds of force. In some embodiments, the predetermined force threshold may be 0.3 pounds of force. In some embodiments, the predetermined force threshold may be 0.2 pounds of force. In some embodiments, the predetermined force threshold may be 0.1 pounds of force. Other configurations and/or values are also contemplated.

In some embodiments, the predetermined translation threshold may be 1.0 centimeters (cm). In some embodiments, the predetermined translation threshold may be 1.5 cm. In some embodiments, the predetermined translation threshold may be 2.0 cm. In some embodiments, the predetermined translation threshold may be 2.5 cm. In some embodiments, the predetermined translation threshold may be 3.0 cm. In some embodiments, the predetermined translation threshold may be 3.5 cm. In some embodiments, the predetermined translation threshold may be 4.0 cm. In some embodiments, the predetermined translation threshold may be 4.5 cm. In some embodiments, the predetermined translation threshold may be 5.0 cm. Other configurations and/or values are also contemplated.

As discussed herein, in some embodiments, the pressure control 376 and/or monitoring device 378 may include and/or emit visual and/or audible alarms when the force exceeds the predetermined maximum force and/or when the force drops below the predetermined force threshold. In some embodiments, the pressure control 376 and/or monitoring device 378 may include and/or emit visual and/or audible alarms when the internal pressure exceeds the preset pressure limit (e.g., Release Pressure (Z)) and/or if the internal pressure drops below the predetermined pressure threshold. In some embodiments, the pressure control 376 and/or monitoring device 378 may include and/or emit visual and/or audible alarms when the Total Pressure (X) exceeds the Release Pressure (Z) and/or if the internal pressure drops below the predetermined pressure threshold. In some embodiments, the pressure control 376 and/or monitoring device 378 may include and/or emit visual and/or audible alarms when the Pouch Pressure (X−Y) exceeds the Release Pressure (Z) and/or if the internal pressure drops below the predetermined pressure threshold.

In some embodiments, the regulator 370, the pressure control 376, and/or the pressure relief valve may release pressure from within the balloon shaft 330 and the inflation lumen when the preset pressure limit (e.g., Release Pressure (Z)) is exceeded. In some embodiments, the regulator 370, the pressure control 376, and/or the pressure relief valve may be configured to instantaneously release pressure from within the balloon shaft 330, the inflatable balloon 332, and/or the inflation lumen when the preset pressure limit (e.g., Release Pressure (Z)) is exceeded. In one example, if the Total Pressure (e.g., X mmHg) is greater than or equal to the Release Pressure (e.g., Z mmHg), the pressure control 376 and/or the regulator 370 may release some or all of the internal pressure within the inflatable balloon 332 and the inflation lumen, thereby deflating the inflatable balloon 332 and reducing and/or removing the pressure and/or force being exerted against the distal end of the upper esophageal pouch 22. In another example, if the Pouch Pressure (e.g., X−Y mmHg) is greater than or equal to the Release Pressure (e.g., Z mmHg), the pressure control 376 and/or the regulator 370 may release some or all of the internal pressure within the inflatable balloon 332 and the inflation lumen, thereby deflating the inflatable balloon 332 and reducing and/or removing the pressure and/or force being exerted against the distal end of the upper esophageal pouch 22.

In some embodiments, the pressure control 376 and/or monitoring device 378 may include and/or emit visual and/or audible alarms when axial translation of the elongate shaft 110 reaches the predetermined translation threshold.

In some embodiments, as the upper esophageal pouch 22 relaxes, grows, and/or stretches, the internal pressure within the inflatable balloon 332 may decrease. By monitoring the internal pressure within the inflatable balloon 332, an attending clinician may be able to identify growth and/or stretching of the upper esophageal pouch 22. In some embodiments, the method may include verifying growth and/or stretching of the upper esophageal pouch 22. In some embodiments, the method may include verifying growth and/or stretching of the upper esophageal pouch 22 via ultrasound. In some embodiments, the method may include verifying growth and/or stretching of the upper esophageal pouch 22 via x-ray. Other imaging methods are also contemplated.

Upon verification of growth of the upper esophageal pouch 22, the method may include actuating the adjustment mechanism and/or rotating the knob 306 relative to the handle 302 to change the internal pressure within the inflatable balloon 332 back to the treatment pressure and/or to adjust the force applied to the distal end of the upper esophageal pouch 22 and thus promote additional growth of the upper esophageal pouch 22.

The process described above may be repeated until the threaded shaft 308 and/or the tubular retainer runs out of available travel within the handle 302. However, the growth and/or stretching process of the upper esophageal pouch 22 of the esophagus 20 may not be complete and/or extensive enough to attach the upper esophageal pouch 22 to the lower esophageal pouch 24 at this time. Accordingly, the medical device 300 may be reset and/or repositioned relative to the patient 10 (e.g., the mouth 12, and/or the distal end of the upper esophageal pouch 22), and the process may begin again and be repeated as necessary.

In some alternative configurations, the medical device 300 may be used in the lower esophageal pouch 24 in a similar manner. Access to the lower esophageal pouch 24 may be made by a different manner, surgically for example, but the medical device 300 may function in largely the same manner as described herein with respect to the upper esophageal pouch 22.

Once sufficient growth and/or stretch of the distal end of the upper esophageal pouch 22 and/or the lower esophageal pouch 24 (if necessary) has occurred, the upper esophageal pouch 22 and the lower esophageal pouch 24 may be joined together surgically in a known manner to reconnect and/or re-establish the esophagus 20 of the patient 10. The medical device 300 and method described herein may provide numerous benefits over other treatment devices and/or methods. For example, the medical device 300 and method described herein may avoid external suture anchors, surgical access required to make adjustments, induced paralysis of the patient, and/or unintended tissue tearing or rupturing associated with other treatment devices and/or methods while permitting earlier treatment of the patient 10 regardless of patient weight and maintaining adequate suction of and/or within the upper esophageal pouch 22 to prevent mucous and/or saliva from entering the trachea, which could lead to potential disruption of adequate respiration. Other benefits are also contemplated and/or likely to be achieved.

The materials that can be used for the various components of the medical device 300 and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the handle, the elongate shaft, the distal nub, the elongate tubular member, the force absorbing member, the adjustment mechanism, the knob, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane (for example, Polyurethane 85A), polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system and/or components thereof in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or components thereof to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vasodilating agents; and agents which interfere with endogenous vasoactive mechanisms.

It should be understood that this disclosure is, in many respects, only illustrative. Changes may be made to details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device for treating esophageal atresia, comprising:
a handle;
an elongate shaft extending distally from the handle, the elongate shaft configured to extend into an esophagus of a patient and into an upper esophageal pouch of the patient, the elongate shaft having a distal nub configured to engage a distal end of the upper esophageal pouch;
a force absorbing member disposed within the handle, the force absorbing member permitting the elongate shaft to translate axially relative to the handle; and
an inflatable balloon disposed within the handle;
wherein an internal pressure within the inflatable balloon increases as the force absorbing member is compressed.

2. The medical device of claim 1, wherein the inflatable balloon cooperates with the force absorbing member to exert a force along the elongate shaft to apply the force to the distal end of the upper esophageal pouch.

3. The medical device of claim 2, further comprising a balloon shaft fixedly attached to the inflatable balloon and having an inflation lumen extending from the inflatable balloon to a regulator disposed outside of the handle.

4. The medical device of claim 3, wherein the regulator is configured to deflate the inflatable balloon when the internal pressure within the inflatable balloon exceeds a preset pressure limit.

5. The medical device of claim 2, wherein the handle includes an adjustment mechanism configured to change the internal pressure within the inflatable balloon.

6. The medical device of claim 5, wherein the adjustment mechanism is configured to change the force applied to the distal end of the upper esophageal pouch.

7. The medical device of claim 5, wherein the adjustment mechanism includes:
a knob configured to rotate relative to the handle; and
a threaded shaft engaged with the knob within the handle;
wherein a first end of the threaded shaft engages a first end of a tubular retainer, the force absorbing member and the inflatable balloon being disposed within the tubular retainer;
wherein rotation of the knob relative to the handle translates the tubular retainer axially within the handle.

8. The medical device of claim 5, wherein the adjustment mechanism is configured to axially translate the inflatable balloon within the handle.

9. The medical device of claim 5, wherein the adjustment mechanism is configured to axially translate the force absorbing member within the handle.

10. The medical device of claim 1, further comprising an elongate tubular member extending distally from the handle;
wherein the distal nub has a first outer diameter;
wherein the elongate tubular member has a second outer diameter less than the first outer diameter;
wherein at least a portion of the elongate shaft is movably disposed within the elongate tubular member.

11. The medical device of claim 10, wherein the elongate tubular member includes a suction lumen configured to be in fluid communication with a source of suction.

12. The medical device of claim 11, wherein the elongate tubular member includes a distal basket configured to receive at least a portion of the distal nub;
wherein the distal basket is configured such that suction within the distal end of the upper esophageal pouch is permitted regardless of a position of the distal nub relative to the distal basket.

13. A medical device for treating esophageal atresia, comprising:
a handle;
an elongate shaft extending distally from the handle, the elongate shaft configured to extend into an esophagus of a patient and into an upper esophageal pouch of the patient, the elongate shaft having a distal nub configured to engage a distal end of the upper esophageal pouch;
a spring disposed within the handle, the spring permitting the elongate shaft to translate axially relative to the handle in response to movements of the patient; and
an inflatable balloon disposed within the handle;
wherein an internal pressure within the inflatable balloon increases as the spring is compressed.

14. The medical device of claim 13, wherein a balloon shaft fixedly attached to the inflatable balloon and including an inflation lumen extending from the handle engages with a longitudinal advancement-retraction mechanism disposed proximal of the handle.

15. The medical device of claim 13, wherein the handle includes a knob configured to rotate relative to the handle to change the internal pressure within the inflatable balloon.

16. The medical device of claim 13, wherein the handle includes a position indicator configured to communicate an axial position of the elongate shaft relative to the handle.

17. A method of treating esophageal atresia, comprising:
preparing a medical device for treating esophageal atresia, the medical device comprising:
a handle including an adjustment mechanism;
an elongate shaft extending distally from the handle, the elongate shaft having a distal nub configured to engage a distal end of an upper esophageal pouch of the patient;
a force absorbing member disposed within the handle and permitting the elongate shaft to translate axially relative to the handle; and
an inflatable balloon disposed within the handle;
inflating the inflatable balloon to an internal pressure, wherein the internal pressure is an initial pressure;
positioning the distal nub of the elongate shaft against the distal end of the upper esophageal pouch;
fixing the medical device in place relative to the distal end of the upper esophageal pouch;
actuating the adjustment mechanism to change the internal pressure within the inflatable balloon to a treatment pressure, wherein the inflatable balloon cooperates with the force absorbing member to exert a force along the elongate shaft to apply the force to the distal end of the upper esophageal pouch;
wherein the force absorbing member is configured to permit the elongate shaft to translate axially relative to the handle in response to patient movements while maintaining the distal nub against the distal end of the upper esophageal pouch.

18. The method of claim 17, wherein the internal pressure within the inflatable balloon increases as the force absorbing member is compressed in response to patient movements.

19. The method of claim 17, wherein an inflation lumen extends from the inflatable balloon to a regulator disposed outside of the handle;
wherein the regulator is configured to deflate the inflatable balloon when the internal pressure within the inflatable balloon exceeds a preset pressure limit.

20. The method of claim 17, further comprising:
monitoring the internal pressure within the inflatable balloon over time;
monitoring axial translation of the elongate shaft over time; and
actuating the adjustment mechanism to increase the internal pressure when the internal pressure drops below a predetermined pressure threshold or when axial translation of the elongate shaft reaches a predetermined translation threshold.
